(19) United States
(12) Patent Application Publication
(10) Pub. No.: US 2003/0190352 A1
(43) Pub. Date: Oct. 9, 2003

(54) Compositions of venlafaxine base

(75) Inventors: Juan Cucala Escoi, Barcelona (ES); Montserrat Gallego Luengo, Barcelona (ES); Marinus J. M. Oosterbaan, Nijmegen (NL); Frantisek Picha, Brno (CZ)

Correspondence Address: FLESHNER & KIM, LLP, P.O. Box 221200, Chantilly, VA, 20153-1200, US

(73) Assignee: SYNTHON BV

(21) Appl. No.: 10/397,353

(22) Filed: Mar. 27, 2003

Related U.S. Application Data
(60) Provisional application No. 60/367,736, filed on Mar. 28, 2002.

(51) Int. Cl. A61K 31/137; A61K 9/20

(52) U.S. Cl. 424/465; 514/649

(57) ABSTRACT

Solid venlafaxine base can be advantageously employed in making pharmaceutical compositions, especially extended release compositions.

This application claims the benefit of priority under 35 U.S.C. §119 from prior U.S. provisional application 60/367,736, filed Mar. 28, 2002, the entire contents of which are incorporated herein by reference.

### BACKGROUND OF THE INVENTION

The present invention relates to pharmaceutical compositions using venlafaxine base, to pharmaceutically useful forms of venlafaxine base and methods of making and using the same.

Venlafaxine is the common name for the compound 1-[2-(dimethylamino)-1-(4-methoxyphenyl) ethyl]cyclohexanol, having the structure shown below.

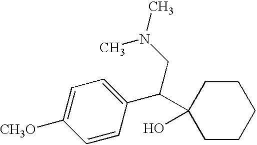

U.S. Pat. No. 4,535,186 describes a class of hydroxycycloalkanephenethyl amines as being useful antidepressants and exemplifies the compound now known as venlafaxine hydrochloride as one of the suitable species. Venlafaxine hydrochloride is approved for sale in various countries including the United States of America. It is available as an immediate release tablet and as an extended release capsule under the brand names EFFEXOR® (Wyeth Ayerst) and EFFEXOR ER® (Wyeth Ayerst), respectively.

Venlafaxine has been the subject of various research endeavors. For example, U.S. Pat. No. 5,043,466 describes a process for making cyclohexanol derivatives in a specified solvent composition. Example 3 of this patent shows the synthesis of venlafaxine as the hydrochloride salt thereof. See also Yardley et al. in J. Med. Chem. 1990, 33 (10), 2899-2905.

U.S. Pat. No. 6,274,171 and related EP 0 797 991A1 disclose encapsulated extended release formulations for venlafaxine hydrochloride. These patents indicate that commercial venlafaxine hydrochloride tablets were administered two or three times daily, but that due to variations in the drug concentration in the patient's blood plasma caused by such a dosing regimen, unwanted side effects, especially nausea and vomiting were common. A once daily, encapsulated extended release dosage form is disclosed that provides a flattened drug plasma profile and reduces these side effects. The encapsulated dosage form is taught to comprise spheroids of venlafaxine hydrochloride, microcrystalline cellulose, and hydroxypropylmethylcellulose (HPMC). These spheroids are coated with a mixture of ethyl cellulose and HPMC. By providing an appropriate amount of the coating, the desired blood plasma profile can be obtained. An acceptable batch of coated spheroids will meet the following in vitro dissolution profile:

| | Average % venlafaxine |
|---|---|
| Time (hours) | hydrochloride released |
| 2 | <30 |
| 4 | 30-55 |
| 8 | 55-80 |
| 12 | 65-90 |
| 24 | >80 |

using USP Apparatus 1 (basket) at 100 rpm in purified water at 37° C. The coated spheroids can be from a single batch or represent a blend of different batches.

U.S. Pat. No. 6,274,171 and EP 0 797 991 also state that forming an extended release dosage from of venlafaxine hydrochloride was difficult in part due to the high water solubility of the hydrochloride salt. In fact, these patents disclose that “[n]umerous attempts to produce extended release tablets by hydrogel technology proved to be fruitless because the compressed tablets were either physically unstable (poor compressibility or capping problems) or dissolved too rapidly in dissolution studies.” See U.S. Pat. No. 6,274,171 at column 4, lines 60-65 and EP 0 797 991 at page 3 lines 35-37. Unlike the encapsulated extended release formulations described in these patents, a hydrogel extended release venlafaxine hydrochloride tablet is taught to typically exhibit a dissolution profile wherein 40%-50% is released at 2 hours, 60%-70% is released at 4 hours, and 85%-100% is released at 8 hours.

WO99/22724 also discloses encapsulated venlafaxine hydrochloride extended release dosage forms. These formulations differ from those in U.S. Pat. No. 6,274,171 and EP 0 797 991 in that the spheroid is substantially free of HPMC. Apparently HPMC can be omitted from the spheroid when smaller amounts of venlafaxine hydrochloride are employed.

Although venlafaxine hydrochloride provides good pharmaceutical activity, it would be beneficial to find other forms of venlafaxine. In particular, venlafaxine forms that are easier to handle would be advantageous. Venlafaxine hydrochloride is relatively aggressive towards handling equipment and is irritating to the skin, etc., of human personnel that handle the pure active. A venlafaxine form that is less aggressive and less irritating would be desirable. It is further desirable to provide a venlafaxine form that can be easily formulated into various dosage forms including extended release tablets.

### SUMMARY OF THE INVENTION

The present invention is based on the discovery that solid forms of venlafaxine base have certain advantageous properties for pharmaceutical formulations. As venlafaxine base is a lower water soluble and lower melting form of venlafaxine, it can be formulated into pharmaceutical compositions that are not obtainable with venlafaxine hydrochloride. Similarly, some venlafaxine base pharmaceutical compositions can be produced by processes that are not suitable for venlafaxine hydrochloride.

Accordingly, a first aspect of the invention relates to a pharmaceutical composition comprising a venlafaxine base and a pharmaceutically acceptable excipient. The composition can be an immediate release dosage form or an extended release dosage form and embraces tablets as well as pellets/beads/spheroids or other encapsulated forms. The extended release tablet or capsule preferably provides sufficient extended release so that once daily dosing is possible. In one embodiment, an extended release composition comprising venlafaxine base and calcium phosphate is preferred.

Another aspect of the invention relates to a venlafaxine composition which comprises venlafaxine base dispersed in a solid carrier. The dispersion can be particles of venlafaxine but more preferably is a molecular dispersion of venlafaxine. Preferred dispersion-forming carriers are polymers and waxes. The solid dispersion, which improves the stability and handling of the venlafaxine base, is a useful form for incorporating venlafaxine, with or without additional excipients, into a pharmaceutical dosage form such as tablets or capsules.

A further aspect of the invention relates to the use of venlafaxine base in treating venlafaxine-treatable diseases or conditions. Hence the invention provides a method for treating a venlafaxine-treatable disease or condition, which comprises administering to a patient in need thereof an effective amount of a pharmaceutical composition comprising venlafaxine base and a pharmaceutically acceptable excipient. The venlafaxine base is typically administered as a tablet or capsule and is preferably administered once daily.

Another aspect of the invention relates to a process, which comprises dispersing venlafaxine base in a liquid-phase carrier and solidifying the liquid phase to form a solid dispersion of venlafaxine base. In one embodiment the dispersing step comprises mixing venlafaxine base and a molten fusible carrier to form a partially melted mass and the solidifying step comprises cooling the partially melted mass to form a solidified product. In another embodiment, the liquid phase carrier comprises a carrier dissolved in a solvent and the solidifying step comprises removing the solvent. The dispersion of the venlafaxine can be a molecular dispersion.

### DETAILED DESCRIPTION OF THE INVENTION

The present invention is based on the surprising discovery that a solid, pharmaceutically useful form of venlafaxine base can be formed. Furthermore, the invention is based on the subsequent discovery that venlafaxine base exhibits low water solubility and slow dissolution in water, which combined with its high loading capabilities, make it an advantageous active for formulating into pharmaceutical dosage forms, especially extended release dosage forms. In addition, venlafaxine base is less aggressive, less irritating, and easier to handle than venlafaxine hydrochloride. Accordingly, venlafaxine base is easier to formulate into a variety of dosage forms, especially extended release dosage forms, than venlafaxine hydrochloride.

“Venlafaxine base” as used herein means the compound 1-[2-(dimethylamino)-1-(4-methoxyphenyl) ethyl]cyclohexanol and includes the racemate or mixture of enantiomers of venlafaxine base as well as the pure or substantially pure (+) or (−) enantiomer of venlafaxine base (hereinafter sometimes referred to as (+)-venlafaxine and (−)-venlafaxine).

A “pharmaceutically useful form” as used herein means that the venlafaxine base is of such a grade as to be suitable for use in a pharmaceutical formulating method and/or for inclusion into a pharmaceutical composition; i.e. ready for such pharmaceutical manufacturing steps as blending, mixing, or milling, etc. The form needs to be susceptible of reliable and reproducible manufacture. As a pharmaceutical active ingredient, the solid venlafaxine base should be relatively pure, typically 5.0 wt % or less, preferably 2.0 wt % or less, more preferably 1.0 wt % or less, still more preferably 0.5 wt % or less of impurities. The solid should be substantially free of solvent, especially ethyl acetate or diethyl ether. Typically the amount of solvent is 3 wt % or less, preferably 1 wt % or less, more preferably 0.5 wt % or less.

The solid form is preferably a white powder. The lack of color in comparison to the prior art evaporate residues indicates that coloring impurities and/or solvents are absent or sufficiently minimized to afford a white color. Preferably the white color meets or exceeds the B9 standard as determined by Pharmacopieal Europe test 2.2.2 (i.e., 0.1% solution in 1% aqueous HCl test).

The solid form is preferably in the form of particles and in particular crystals. The particles are preferably “filtratable,” meaning that the particles can be separated from a solvent by use of a filter. Generally, the particles must be at least 0.5 microns on average to be filtratable. The particle population typically has an average particle size within the range of 0.5 to 200 microns, more typically 10 to 100 microns. In some embodiments, such as for use in a wet granulation or melt granulation processes, the average particle size is preferably within the range of 1 to 50 microns, more typically 5 to 50 microns, and preferably 10 to 50 microns. In other embodiments, such as for use in direct compression tabletting processes, the average particle size is within the range of 50 to 150 microns, more typically 50 to 100 microns.

In some cases, e.g. for making relatively concentrated tablet compositions comprising at least 30% of the venlafaxine base by direct compression, it is even desirable to have venlafaxine base particles of an average particle size of at least 200 microns, preferably 250-500 microns.

In terms of bulk density, the population of solid venlafaxine base particles should preferably exhibit the bulk density greater than 0.4 mg/ml, preferably 0.4-0.8 mg/ml.

Conveniently the solid venlafaxine base is a precipitate, especially a filtratable precipitate. By precipitating venlafaxine base from a venlafaxine solution, a solid form is obtained that is relatively pure and, after isolation from the liquid, typically of white color. Such a solid is preferably of such quality as to be a pharmaceutically useful form. The precipitation according to the present invention is preferably “induced” in that a contrasolvent or seeding crystal, or both, is present/added to cause precipitation instead of merely decreasing temperature or solvent volume, albeit such steps may and usually are additionally carried out as well. As used herein, the term “venlafaxine solution” means any solution that contains a venlafaxine moiety or ion thereof and specially includes venlafaxine salt solutions as well as crude venlafaxine base solutions including the reaction medium obtained by synthesizing venlafaxine. The venlafaxine solution contains a venlafaxine solvent sufficient to dissolve the venlafaxine. Generally organic polar solvents are suitable to dissolve venlafaxine. Specific examples include esters such as ethyl acetate, ethers such as diethyl ether, ketones such as acetone, and lower alcohols such as methanol, ethanol, and 1- or 2-propanol. Contrasolvents are liquids in which venlafaxine base is less soluble than the solvent. Examples include water and aliphatic or alicyclic hydrocarbon solvents such as hexane, heptane, petroleum ether, and cyclohexane. The following combinations are preferred, ethyl acetate solvent and n-heptane contrasolvent; ethanol solvent and n-heptane contrasolvent; and ethanol as solvent and water as a contrasolvent. In the combinations comprising water, it is also preferred that a venlafaxine base seeding crystal is added.

Generally the temperature of the solution is decreased before and/or during precipitation. While the rate of decrease is not particularly limited, it can have an effect on the size of the particles produced. It is preferred to use cooling rates of not greater than 20° C./hour, more preferably about 10° C./hour, in order to make large crystal sizes such as 200 microns or greater.

The venlafaxine base is precipitated from the venlafaxine solution to form a suspension, distribution, and/or slurry of solid venlafaxine base in the remaining liquid. Preferably the solid venlafaxine is homogeneous in that the solid particles are of relatively uniform size, i.e. at least 50%, more preferably at least 60%, of the particles are within the range of +/−25 microns from the average particle size. The solid particles, normally crystals, are preferably separated from the solution by filtration. The particles can be washed and/or dried. If desired, the particles can be re-precipitated by dissolving them in a venlafaxine solvent to form a solution and carrying out induced precipitation again.

The venlafaxine solution can be formed by a variety of ways and is not particularly limited in this respect. One route to obtaining a venlafaxine solution involves forming a solution of a venlafaxine salt such as venlafaxine hydrochloride. The venlafaxine salt solution can be neutralized by adding an organic or inorganic base, typically NaOH, to form a venlafaxine base solution before inducing precipitation. Another way to form a venlafaxine solution is from a crude venlafaxine base. The crude venlafaxine solution can be made by dissolving in a venlafaxine solvent a previously formed solid venlafaxine base, especially non-precipitated solid venlafaxine formed by evaporation of the solvent. Alternatively, the crude venlafaxine solution can be the extracted venlafaxine product from a reaction medium. Further, the crude venlafaxine solution can be the entire reaction medium resulting from the synthesis of venlafaxine.

The venlafaxine base used in the present invention can be in a variety of forms including crystalline and non-crystalline forms. Crystalline forms include anhydrous and solvated, particularly hydrated, forms. Examples are venlafaxine hemihydrate, monohydrate, dihydrate, trihydrate, ethanol solvate, ethyl acetate solvate, acetone solvate or hexane solvate. The hydrates or solvates may be converted to preferred solvent-free or anhydrous forms of venlafaxine base by conventional methods, e.g. by drying at reduced pressure. Furthermore, any polymorphic modification of anhydrous venlafaxine base can be used in the present invention. Non-crystalline forms include amorphous forms as well as molecular dispersions.

The solid, pharmaceutically useful venlafaxine base of the present invention is not limited to precipitates made by the above described induced precipitation method. Rather, the solid venlafaxine base of the present invention can be made by whatever means or source, provided that the solid is of sufficient quality to meet or exceed pharmaceutical standards. For examples, solids made by evaporating off a solvent to form a colored residue, followed by, inter alia, washing, drying and/or milling steps, are equally a part of the present invention provided that the solid possesses the necessary quality, i.e. it is a pharmaceutically useful solid of venlafaxine base.

Solid venlafaxine base can advantageously be incorporated into a pharmaceutical composition by combining it with at least one pharmaceutically acceptable excipient. The pharmaceutical compositions of the present invention include the unit dosage form as well as the intermediate bulk formulations such as pellets, beads, powder blends, etc. Typically the composition is a finished dosage form also referred to as a unit dose. Dosage forms include oral dosage forms, topical dosage forms such as a transdermal patch, parenteral dosage forms such as an injectable solution, and rectal dosage forms such as a suppository, but is not limited thereto. Oral dosage forms are the most preferred due to the ease of administration and include solid oral dosage forms such as capsules, tablets, sachets/granules, and powders, as well as liquid oral dosage forms such as solutions, suspensions, and emulsions. Most preferred are solid oral dosage forms.

Pharmaceutically acceptable excipients are well known in the art and include diluents, fillers, binders, lubricants, disintegrants, glidants, colorants, pigments, taste masking agents, sweeteners, plasticizers, and any acceptable auxiliary substances such as absorption enhancers, penetration enhancers, surfactants, co-surfactants, and specialized oils. The proper excipient(s) are selected based in part on the dosage form, the intended mode of administration, the intended release rate, and manufacturing reliability. Examples of common types of excipients include various polymers, waxes, calcium phosphates, and sugars. Polymers include cellulose and cellulose derivatives such as HPMC, hydroxypropyl cellulose, hydroxyethyl cellulose, microcrystalline cellulose, carboxymethylcellulose, sodium carboxymethylcellulose, calcium carboxymethylcellulose, and ethylcellulose; polyvinylpyrrolidones; polyethylenoxides; and polyacrylic acids including their copolymers and crosslinked polymers thereof, i.e. Carbopol® (B.F. Goodrich), Eudragit® (Rohm), polycarbophil and chitosan polymers. Waxes include white beeswax, microcrystalline wax, carnauba wax, hydrogenated castor oil, glyceryl behenate, glycerylpalmito stearate, saturated polyglycolyzed glycerate. Calcium phosphates include dibasic calcium phosphate, anhydrous dibasic calcium phosphate, and tribasic calcium phosphate. Sugars include simple sugars such as lactose, maltose, mannitol, fructose, sorbitol, sacarose, xylitol, isomaltose, and glucose as well as complex sugars (polysaccharides) such as maltodextrin, amylodextrin, starches, and modified starches.

The venlafaxine base used in the pharmaceutical compositions of the present invention means any form of venlafaxine, derived from whatever source. However, the quality of the base form must not be inconsistent with forming a pharmaceutical composition. When solid venlafaxine base is used in the composition, it is preferably one of the above-described pharmaceutically useful forms such as a white powder, precipitate, etc. but is not limited thereto.

Alternatively, a dispersion of venlafaxine base in a solid carrier can be a convenient way to incorporate venlafaxine into a dosage form. The dispersion is preferably of such a quality that it is a molecular dispersion of venlafaxine base, although such is not required. A molecular dispersion of venlafaxine is a non-crystalline form; i.e. the venlafaxine is not in crystalline form within the solid excipient.

In the venlafaxine dispersion, the solid matrix-forming carrier is generally a polymer or a wax, especially a fatty acid wax.

Specifically, suitable polymers include polyvinylpyrrolidone, polyethylene glycol, polyoxyethylene derivatives, hydroxypropyl methylcellulose, ethyl cellulose, polyacrylic/methacrylic acids esters, etc. Dispersions comprising water soluble polymers may improve release rate and/or bioavailability of venlafaxine in body fluids while dispersions comprising insoluble polymers may accordingly suitably retard the release of venlafaxine.

Suitable waxes include fatty acid waxes comprising a C10-C24, particularly C16-C22, aliphatic monocarboxylic acid and/or an ester thereof with mono- or polyfunctional alcohol. Even more specifically, the ester-forming alcohol is glycerol. The preferred fatty acid wax comprises glyceryl palmitostearate or glyceryl behenate.

The mass ratio of venlafaxine to the carrier in the dispersion may vary, for instance from 5:95 to 95:5, more typically 1:10 to 10:1. In some embodiments, especially with polymers, the ratio is preferably approximately 1:1.

Such dispersion may be made by dispersing venlafaxine base in a liquid phase carrier and then solidifying the liquid phase. The liquid phase carrier can be a melt of the carrier or a solution of the carrier in a solvent. Solidification can involve cooling to solidify the molten mass or removing the solvent to leave a solid. Thus, venlafaxine can be dispersed in a solid carrier by dissolving the venlafaxine and a carrier in a solvent and evaporating of the solvent, or by co-melting venlafaxine base with the carrier and cooling the melt, or by combination of these techniques. The co-melting process advantageously can comprise hot melt granulation, which is described in more detail hereinafter, or melt extrusion. It should be noted that the co-melting need not be complete in that the venlafaxine, is not required to melt, so long as the matrix-forming carrier is sufficiently melted or fluid so as to allow mixing and dispersing of the venlafaxine therein. Preferably, however, the venlafaxine is also melted so that a true molecular dispersion is formed upon solidification.

A typical example is a solid dispersion of venlafaxine base in polyvinylpyrrolidone, which is advantageously prepared by dissolving venlafaxine and polyvinylpyrrolidone in ethanol and evaporating the solvent. The resulting product is a solid material in which the venlafaxine base is dispersed. It can be in the physical form of particles or granules and/or it can be milled or chopped to form a powder or granule form. Such physical forms of the dispersion are useful for creating tablets, pellets or other dosage forms.

By making solid venlafaxine base in a form of a solid dispersion, physical characteristics and stability characteristics of venlafaxine may be improved. For instance, a dispersion of venlafaxine in polyvinylpyrrolidone can aid in tabletting performance while a dispersion of venlafaxine in a wax can aid in controlling the release of the drug, etc.

The amount of venlafaxine base contained in a unit dosage form is an amount effective to treat one or more venlafaxine-treatable diseases or conditions as is hereinafter defined and can be determined by workers skilled in the art without undue experimentation. Generally this amount ranges from 2 mg to 300 mg. For oral dosage forms the amount is generally from 30 mg to 300 mg per unit dose. Contemplated doses include amounts of about 37.5 mg, 75 mg, 100 mg, 112.5 mg, 150 mg, 200 mg, and 300 mg strengths. Because the free base is used and not a salt thereof, the actual weight of the active ingredient is less in the present invention as compared with venlafaxine HCl, for example. This allows for higher loading of the active, i.e. more drug per weight of excipients, and therefore a lower amount of excipients are needed. Accordingly, a smaller dosage form is one advantage of using venlafaxine base. Preferably the dosage form contains at least 40 wt %, more preferably at least 50 wt % of the venlafaxine base.

As mentioned above, oral dosage forms are preferred and include tablets, capsules, sachets/granules, and powders. Tablets can be soluble tablets, dispersible tablets, effervescent tablets, chewable tablets, lyophilized tablets, coated tablets including sugar coatings, enteric coatings, and gastro-soluble coatings, and modified release tablets including microencapsulated active substance tablets, matrix tablets, and coated tablets such as polymer coated extended release tablets and osmotic tablets of the mono-compartmental or bi-compartmental type. Capsules include hard gelatin capsules that can be filled with powder, pellets, granules, small tablets or mini-tablets. The capsule and/or the material placed within can be coated such as for enteric release or modified release. Soft capsules are also included and are more typically filled with liquids or dispersions, but are not limited thereto. Sachets or granules can be effervescent granules, coated granules, enteric granules, or modified release granules.

One embodiment of the present invention relates to an immediate release tablet. An “immediate release” as used herein means that at least 80% of the venlafaxine in the tablet is dissolved by 30 minutes under a dissolution test using USP Apparatus 1 (basket) at 100 rpm in purified water at 37° C. Any conventional immediate release composition can be used in formulating the venlafaxine base immediate release tablet. Typically such tablets contain one or more binders and/or diluents such as HPMC, microcrystalline cellulose, a calcium phosphate, lactose, and mannitol; a lubricant such as magnesium stearate; and optionally a disintegrant such as sodium starch glycollate, crosscarmellose or crosspovidone. Additional excipients such as colorants, antioxidants, etc can also be present.

More preferably, however, the solid oral dosage form is an extended release dosage form. This can be accomplished in either a tablet or a capsule form. An extended release dosage form as used herein means that in a dissolution test using USP Apparatus 1 (basket) at 100 rpm in purified water at 37° C., less than 80% of the venlafaxine base is dissolved during the first two hours, more typically less than 50%, and preferably less than 30% of the venlafaxine base is dissolved during the first two hours. Extended release tablets or capsules generally allow for twice a day, or more preferably once a day dosing, to provide 24 hour therapeutic blood plasma levels of venlafaxine to the patient. In this regard, the most preferred dosage form is one which provides once daily dosing. Such a composition should meet the following in vitro dissolution profile:

| Time (hours) | Average % venlafaxine released |
|---|---|
| 2 | <30 |
| 4 | 30-55 |
| 8 | 55-80 |
| 12 | 65-90 |
| 24 | >80 |

using USP Apparatus 1 (basket) at 100 rpm in purified water at 37° C. Most advantageously the extended release dosage from meets the above dissolution profile also in 0.1N HCl aqueous solution.

Preferably, an extended release dosage form meets the above dissolution profile using a two media dissolution test. Specifically, during the first two hours, the media is a simulated gastric fluid (SGF) of pH 1.2 while during the remaining hours the media is a simulated intestinal fluid (SIF) of pH 6.8. This two media test can provide more accurate predictions of in vivo performance in some circumstances, especially when an enteric coating is present on the dosage form.

For purposes of the present invention, the simulated fluids are defined as follows:

SGF (USP Simulated Gastric Fluid without pepsin) composition:

| | | |
|---|---|---|
| HCl | qs | pH 1.2 |
| NaCl | | 0.2% |
| water | qs | 1000 ml |

SIF (USP Simulated Intestinal Fluid without pancreatin) composition:

| | | | |
|---|---|---|---|
| KH₂PO₄ | | 6.8 | g |
| NaOH | qs | pH | 6.8 |
| water | qs | 1000 | ml |

In terms of in vivo performance, the extended release venlafaxine base pharmaceutical composition according to the present invention preferably exhibits on average a maximum venlafaxine blood plasma level not earlier than 4 hours, more preferably not earlier than 6 hours after administration of the composition. Typically the average peak plasma level is reached between 4 and 10 hours, more preferably between 6 and 8 hours after administration. In this regard, a preferred composition is bioequivalent to the commercially available EFFEXOR XR®.

Extended release tablets can be formulated according to any of the known techniques such as those based on matrix technology, osmotic pressure technology, multiparticulates compressed into tablets, multilayer tablets having at least one layer based on one of the foregoing, as well as coated tablets, using known materials and methods.

Tablets employing a matrix, in either a monolithic tablet or in one or more layers optionally built on a non-pareil seed or tablet core, are generally the most common and frequently the easiest to form from a commercial manufacturing standpoint. The matrix provides a diffusion and/or erosion release of the drug. The matrix is generally composed of at least one type of matrix material selected from hydrophilic (hydrogel), inert, lipophilic, and biodegradable matrix materials. Materials used for each of these kinds of matrices in pharmaceutical oral dosage forms are well known in the art and are briefly described below.

A hydrophilic matrix material is generally a polymeric material that swells upon contact with water to form a diffusion barrier. Suitable materials include cellulose derivatives such as methylcelluloses (i.e. having a viscosity of 400 cP to 4000 cP), hydroxyethylcellulose, HPMC, and sodium carboxymethyl cellulose; polysaccharides such as galactomannanes, potassium alginates, sodium alginates, agar-agar, carrageen, arabic gum, and sterculia gum; polyacrylates such as CARBOPOL 934, EUDRAGIT LD 35; Noveon or polycarbophils; and other water swellable polymers such as polyvinyl alcohol.

Inert matrix materials provide a tortuous path for the drug to escape the dosage form thereby controlling diffusion of the drug. Such materials include ethylcellulose (ETHOCEL).

Lipophilic matrix materials work through a combination of erosion and diffusion. Examples of lipophilic materials include glyceryl palmitosterate (PRECIROL ATO 5), glyceryl behenate (COMPRITOL 888 ATO) and Hydrogenated castor oil (CUTINA HR).

Biodegradable matrix materials also operate through a combination of erosion and diffusion. Biodegradable materials include, for example, polyesters of lactic acid and glycolic acid, polyorthoesters, polyanhydrides and caprolactones. A further description of this technology is set forth in WO02/11701, WO92/04013, and EP 1 005 863.

It has been discovered that, due to physical properties of venlafaxine base in terms of water solubility, an extended release tablet can be formed by simple techniques. In general, an otherwise immediate release tablet formulation, minus any disintegrant, provides for a multi-hour release of venlafaxine in water. Accordingly, an enteric coated tablet will provide for extended release of venlafaxine. Generally no special extended release coating or matrix is needed to maintain the extended release in water as the insolubility of the venlafaxine base provides for slow release. Of course such release modifying agents can be incorporated if desired. Surprisingly, venlafaxine base can even be formulated into a once-daily extended release tablet in water by such a simple design. Moreover, immediate release venlafaxine base tablets can also be extended release coated with any extended/modified release film to obtain an extended release profile in different media.

Preferably, the tablet core contains one or more of the excipients identified above for an immediate release tablet, except a disintegrant, which should normally be excluded. Generally, the tablet of the present invention comprises 10%-70% of a venlafaxine base, and 90% to 35% of excipients such as a diluent, carrier or filler. In some embodiments it may be advantageous for the weight ratio of venlafaxine base to excipients to be in the range of 0.8-1.2:1, preferably approximately 1:1, respectively. Suitable fillers are, e.g. calcium hydrogenphosphate, microcrystalline cellulose or lactose, suitable lubricants are magnesium stearate, precirol, sodium stearyl fumarate (Pruv) or talc.

Surprisingly, very advantageous extended release profiles of a venlafaxine tablet may be obtained by simple blending and compressing (e.g. by means of direct compression) of a mixture of venlafaxine base and calcium hydrogenphosphate. Although no traditional extended release excipient or coating is present, such a tablet unexpectedly provides an extended release profile. This advantageous release profile may be obtained both with anhydrous and with hydrated phosphates and it is independent on the brand, particularly on the pH of the phosphate. In some embodiments a lipophilic matrix material such as a wax, especially glyceryl behenate, can be mixed with the calcium hydrogenphosphate to provide an improved extended release profile. The amount of lipophilic matrix material is generally up to 50%, normally 10% to 30%. The amount of calcium hydrogenphosphate and lipophilic matrix are generally in a ratio of 10:0.1 to 20, preferably 10:1 to 11, respectively, and the total amount of these two excipients is typically 30% to 90%, more preferably 40% to 70% of the tablet or dosage form. Such a combination together with venlafaxine base, which preferably accounts for 95% or more, preferably 98%, of the tablet, can be blended and tabletted to form an extended release tablet. The remaining tablet weight, if any, typically includes a lubricant.

From practical reasons, it is also advantageous first to granulate venlafaxine base with a small amount of a hydrophilic polymer, for instance with polyvinylpyrrolidone and then to blend and compress the granulate with calcium phosphate. The release profile is not basically affected, but the tabletting properties of venlafaxine base (which is obtainable in very fine particle size) for the compression step are improved. Moreover, such composition allows a reduction in the amount of the filler and thus enhances the loading capacity of venlafaxine. In this embodiment, the relative content of venlafaxine in the tablet may be more than 50%, which allows for the preparation of extended-release minitablets.

In another embodiment, the venlafaxine base is used in the form of large particle sizes such as greater than 100 microns, preferably greater than 200 microns and generally in the range of 250 to 500 microns. Such a venlafaxine can be blended with the calcium phosphate and optionally additional excipients and tabletted by direct compression.

To avoid too rapid disintegration of the tablet comprising calcium phosphate, the tablet composition may be improved by adding a binder. Hydrophilic or lipophilic binders are both possible. A suitable binder is, for instance, glyceryl behenate.

As mentioned previously, it is preferred that an extended release dosage meets the above dissolution profile also in simulated intestinal fluid (SIF) of pH 6.8 medium, because this medium test can provide more accurate predictions of in vivo performance in some circumstances.

An extended release composition in simulated intestinal media of venlafaxine base could be obtained using a lipophilic matrix. The relatively low water solubility and relatively low melting point of venlafaxine base affords the formation of an effective extended release dosage form using a lipophilic matrix. A lipophilic matrix is one that contains a lipophilic excipient and forms an inert porous matrix through which the drug dissolves. An example of such lipophilic matrix is the solid dispersion of venlafaxine base with a wax described above. Preferably a lipophilic matrix-based tablet is formed by hot melt granulation techniques, as is described in more detail hereinafter, wherein the lipophilic matrix material is a fusible carrier. Any of the above identified lipophilic matrix materials can be used as a fusible carrier in a hot melt granulation. The lipophilic matrix-based tablets generally contain 10% to 80% venlafaxine base and 5% to 50%, more typically 8% to 30% of the lipophilic matrix material. In some embodiments it is preferable to have a weight ratio of venlafaxine base to lipophilic matrix material within the range of 1:0.05-0.7, more preferably approximately 1:0.5, respectively. In addition to the lipophilic matrix material, the tablet can further contain additional suitable excipients as described above. Typically the tablet will contain a filler such as calcium phosphate, microcrystalline cellulose, and/or lactose and a lubricant such as magnesium stearate or talc, but is not limited to these excipients. In some embodiments, especially where granulates containing at least the lipophilic matrix and venlafaxine base are used, the tablet can contain wax such as glyceryl behenate, glyceryl palmitostearate or hydrogenated castor oil as a filler. Such extra-granulate wax can also assist in avoiding an initial fast dissolution release. The tablet can be enteric film coated in order to avoid the active substance from being released to the stomach, or film coated with some polymer or mixture of polymer pH independent in order to decrease the initial fast release typical of matrix-based tablets. Another option for controlling/limiting the release of the venlafaxine base into the gastric fluid is to make an enteric coating with a pore forming agent therein. The tablets based on lipophilic matrix materials are preferably once daily dose tablets.

The tablets of venlafaxine base according to the present invention can be made by any known tabletting technique. Suitable techniques include direct compression, dry granulation, wet granulation and hot melt granulation. The compression methods that do not employ a solvent (“dry processes”) are generally preferable.

In general, dry granulation procedures comprise mixing the solid excipients (except lubricants), compacting the mixture in a compactor (e.g. a roller compactor), or double compression, milling the compacted mass, screening the milled granules, mixing with a lubricant and compressing the mixture into tablets. Direct compression procedures generally comprise mixing the solid excipients in one or more stages and compressing the uniform mixture into tablets. After tablet formation, the tablets may optionally be coated. The venlafaxine is normally present in the first mixing step and can be used per se or as a dispersion in a solid excipient.

In addition to wet and dry granulation, hot melt granulation is also suitable for making venlafaxine base pharmaceutical compositions. Hot melt granulation, which is a technique that results in a dispersion of venlafaxine in a solid excipient, generally comprises mixing a fusible carrier in a molten state with venlafaxine base to form a partially melted mass and then cooling the mass to form a solidified product. A fusible carrier is any material that can serve as a binder, carrier or matrix having a melting point within the range of 35° C. to 250° C. Preferably the fusible carrier is lipophilic, i.e., a lipophilic matrix material, and has a melting point within the range of 50° C. to 150° C. As explained above, the preferred product of the hot-melt granulation with a lipophilic carrier is a solid dispersion of venlafaxine base in the lipophilic matrix, i.e. carrier.

Typically venlafaxine base, fusible carrier, and optionally one or more fillers, antiadherent agents, lubricants, etc. are combined or mixed in a granulator. The materials are then heated by any convenient technique, such as by a heating jacket, microwaves, infrared, etc. or a combination of two or more techniques. The mixture is heated to a temperature near or above the melting or softening point of the fusible carrier, thereby allowing the fusible carrier to act as a liquid binder. If two or more fusible carriers are present, such as two lipophilic matrix forming materials, the temperature need only reach near or above the melting or softening point for one of the carriers. This state is considered a “molten” state for purposes of the present invention. The venlafaxine base does not have to melt during this heating or mixing step. The molten fusible carrier is mixed with the venlafaxine base and any additional optionally combined excipients to form a partially melted mass. For clarity, the partially melted mass means that the mass is at least partially melted and includes a full melted or flowable mass. Preferably the mixing is sufficient, given the degree of melting/fluid state of the carrier, the relative amounts of the carrier, the venlafaxine base, and any other excipients present, to form a substantially uniform mixture of the combined ingredients. The mixing is preferably carried out using an impeller and a chopper (stirring blades). While the ingredients are normally combined in a non-molten state and mixed before as well as during the heating step, such is not required. For example, the fusible carrier in molten state can be directly combined and mixed with the venlafaxine base. Similarly, the mixing may begin only after the fusible carrier starts to soften, partially melt, or completely liquefy.

Once mixed into the desired partially melted mass, the mass is cooled to form a solidified product. The cooling can be passive, i.e. by removing the heat source, but more typically involves applying a cooling technique such as cool water through a jacket surrounding the granulation bowl and/or with gas transmission through the bowl mass. The solidified product can be in the form a granules or larger in size such as pellets. Alternatively, the solidified product may comprise much larger pieces such that milling is required to obtain powder or granules. The partially melted mass is preferably also mixed during the cooling step. As in the first mixing step, the mixing preferably is carried out in a granulator using an impeller and a chopper (stirring blades). Mixing during the cooling serves to break up the solidifying mass and aids in the production of granules or pellets.

The solidified product is then converted to a tablet by techniques known in the art. Typically the solidified product is sieved or milled and sieved, optionally combined with one or more additional excipients such as a lubricant and then compressed into tablets. In preferred embodiments, at least the lubricant and more preferably all additional excipients, if any, are mixed with the solidified product to form a tabletting mixture. The tabletting mixture is compressed into tablets. Alternatively, it is possible for all the tablet ingredients to be present in the solidified product.

The tablets made from a hot melt granulation technique are preferably lipophilic matrix-based tablets, more preferably they form extended release tablets as discussed above. In any event, the hot melt granulation tablet preferably has a release profile that satisfies the following:

| | Average % venlafaxine base |
|---|---|
| Time (hours) | released |
| 2 | <30 |
| 4 | 30-55 |
| 8 | 55-80 |
| 12 | 65-90 |
| 24 | >80 |

using USP Apparatus 1 (basket) at 100 rpm in purified water at 37° C. Most preferably the tablets are once daily dose tablets. The extended release tablets preferably have an enteric or functional coating in order to avoid release of the drug in the stomach.

As mentioned above, the venlafaxine base tablets of the invention may be coated. Typically the coating provides suitable gastro-resistance to delay the onset of release of the active component from the tablet. Examples of coating material for gastro-resistant coatings are cellulose acetate phthalate (CAP) (Aquacoat CPD™), co-processed polyvinyl acetate phthalate (PVAP) (Suretetic™), cellulose acetate trimellitate (CAT), acrylic-methacrylic acid copolymers (Eudragit type polymers) (Acryl-EZE™), hydroxypropyl methylcellulose phthalate (HP), hydroxypropyl methyl cellulose acetate succinate (HPMCAS). Such an enteric coating is useful in matrix-based tablets because of the tendency of such tablets to have an initial burst effect due to the presence of the active on or near the surface of the tablet.

Enteric coating prevents from the immediate dissolution of venlafaxine base in acid media. In order to provide an initial immediate dose of venlafaxine, an outer layer of venlafaxine can be formed from any pharmaceutically acceptable excipient and venlafaxine base or acid addition salt thereof. The relative amount of venlafaxine is normally 20% or less, more typically 10% or less, of the total amount of venlafaxine in the tablet.

Another technique that can be used to control the initial burst effect is to make an enteric coating which contains a pore forming agent. A pore forming agent is an excipient that is soluble in acid media, gastric environment. In this way, the pore forming agent is dissolved out of the enteric coating layer by the gastric fluid, thereby leaving a pore by which the drug can be released by a small extent. The remainder of the enteric layer is removed in the intestinal tract to allow release of the remainder of the drug.

In addition to, or instead enteric coating, the tablet can be provided with pH buffering agent and/or bases to provide acid resistance and prevent immediate release in the stomach.

The tablets can be of any size and shape. In one preferred embodiment the tablets are small or mini-tablets in size. Small tablets have a diameter of 3-6 mm while mini-tablets have a diameter of 1-3 mm. One or more of the tablets can be taken as such or, more preferably one or more are loaded into a single capsule to provide a unit dose. Most preferably, the small or mini-tablets provide additive amounts of the venlafaxine base without modifying the release profile. For example, by making a round small tablet of diameter 5 to 6 mm and containing 37.5 mg of venlafaxine, capsules containing 37.5 mg, 75 mg, and 150 mg of venlafaxine base can be formed by filling a standard capsule with 1, 2, or 4 of the small tablets, respectively. By using small tablets in a single capsule, only one tablet formulation and shape is needed to produce multiple dosage strengths. Typically a small or mini-tablet contains 5 to 50 mg of venlafaxine base, especially 10, 25, 30, 37.5, 40, and 50 mg. Depending on the size of the tablet and the capsule from 1 to 10 or more small or mini-tablets can be placed in the capsule.

In addition to filling capsules with small or mini-tablets, an extended release capsule can be formed by filling it with more traditional pellets, beads, and/or spheres. The pellets can be coated with an extended release coating or composition. In addition, different populations of coated pellets can be used in a single capsule, each providing a different release characteristic so that the aggregate release is sustained over a long period; i.e. 12 to 24 hours. Alternatively, the bead population can be substantially homogeneous. A preferred capsule of the pellet type is described in the above-mentioned U.S. Pat. No. 6,274,171 and related EP 0 797 991A1 wherein the venlafaxine hydrochloride used in these patents is replaced with the venlafaxine base of the present invention.

The venlafaxine base of the present invention can be used to treat any disease or condition that is treatable by venlafaxine. A venlafaxine-treatable disease or condition is one that could be improved by a serotonin or norepinephrine uptake inhibitor and specifically includes, without limitation, depressions, panic disorder, generalized anxiety disorder, obesity, post-traumatic stress disorder, late luteal phase dysphoric disorder, attention deficit disorders, Gilles de la Tourette syndrome, bulimia nervosa, and Shy Drager syndrome. See published US patent application US 2001/0012855 A1 for a description of the uses of venlafaxine and salts thereof The venlafaxine base of the present invention can be used to treat such conditions by administering an effective amount to a patient in need thereof. An effective amount is generally known in the art and/or determined using routine skill. Typically the effective amount for a human is 30 to 300 mg of venlafaxine per day. The patients used herein include human and non-human mammals such as dogs, cats, and horses. The route of administration is not particularly limited and includes peroral, parenteral, and transdermal administration. Preferably, the venlafaxine base is administered orally via one or two unit dosage forms, especially extended release tablets or capsules, as described above.

The entire disclosure in each of the patents mentioned in the above description is incorporated herein by reference. The invention will be further described with reference to the following non-limiting examples.

### EXAMPLE 1A

In a 100 ml flask, equipped with a stirring bar,

3.0 g racemic venlafaxine HCl was dissolved in

8 ml water. Then,

0.382 g sodium hydroxide (1 equivalent) was added in one portion. Immediately, an oil separated from the reaction mixture.

8 ml ethanol was added and the resulting mixture was seeded with a few crystals After a few minutes, the oil had solidified, but stirring was continued for 1 hour. The crystals were isolated by filtration, washed with 100 ml of a mixture of ethanol/water (¼), dried overnight at 40° C. under vacuum.

Yield: 2.276 g (86.9%).

mp (76-78° C.), Karl-Fischer (no water).

The solid state properties correspond to venlafaxine base Form I

### EXAMPLE 1B

In a 100 ml flask (closed),

2 g venlafaxine base was heated using an oil bath to a temperature of 90-120° C. venlafaxine melted rapidly (within a few minutes). The flask was kept in the oil bath for another 5 minutes. Then, the flask was stored at room temperature. Initially, a colorless glassy substance was formed, which upon standing at room temperature changed into a white crystalline material.

IR: corresponds with Form II

### EXAMPLE 1C

In a 500 ml flask,

10 g venlafaxine base was dissolved in

25 ml ethanol, filtered prior to addition, and added dropwise to

200 ml water at 70° C. while stirring mechanically. During and after addition, seed crystal of venlafaxine Form II was added in trace amounts. After complete addition of the ethanol solution, the temperature of the oil bath was lowered to 50° C. At this temperature, crystallization occurred. After an additional hour at 50° C., the crystals were isolated by filtration and dried over P₂O₅ at 25° C. overnight.

Isolated yield: 9.312 g (93%).

IR: corresponds to Form II

DSC: single peak at 82.61° C.

### EXAMPLE 2

Round tablets, 5 mm in diameter, containing 44.44% and 50% venlafaxine were made with the following nominal compositions:

| Ingredients | Mg/tablet | Mg/tablet |
|---|---|---|
| Venlafaxine base | 40.00 | 50.00 |
| Microcrystalline cellulose | 33.00 | 33.00 |
| (Avicel PH102) | | |
| Lactose monohydrate for | 16.00 | 16.00 |
| direct compression | | |
| Magnesium stearate | 1.00 | 1.00 |

In SGF, 94.5% and 80.8% of the venlafaxine was dissolved in 15 minutes for the 44.44% tablet and 50% tablet, respectively. However, in water, at one hour only 32.3% and 22.6%, respectively, had dissolved and for both tablets 80% dissolution was not reached until after 4 hours.

### EXAMPLE 3

Tablets were made having the following nominal composition

| | Ratio | Ratio | Ratio |
|---|---|---|---|
| Ingredients | 1:0.5 | 1:0.75 | 1:1 |
| Venlafaxine base | 24.00 | 24.00 | 24.00 |
| HPMC (Methocel K 4M EP) | 12.00 | 18.00 | 24.00 |
| Microcrystalline cellulose (Avicel PH 102) | 48.00 | 48.00 | 48.00 |
| Magnesium stearate | 1.00 | 1.00 | 1.00 |

In SGF, 74.6%, 66.0% and 63.1% of the venlafaxine was dissolved in two hours for the 1:0.5 tablet, 1:0.75 tablet and 1:1 tablet, respectively. However, in water, at two hour only 26.3%, 9.0% and 7.8%, respectively, had dissolved and for these tablets 80% dissolution was not reached until after 4 hours.

### EXAMPLE 4

Tablet composition comprising venlafaxine base (Immediate release composition)

| | | |
|---|---|---|
| Venlafaxine base | 30.00 mg | Active substance |
| Silicified microcrystalline cellulose | 15.00 mg | Filler |
| Anhydrous dicalcium phosphate | 44.10 mg | Filler |
| Magnesium stearate | 0.90 mg | Lubricant |
| Total weight | 90.00 mg | |

Modus operandi: Direct compression

Dissolution rate (24 hours in water, 37° C., basket 100 rpm) was determined by UV spectrophotometry and is expressed in % of the declared amount.

| Hours | % dissolved | Target % dissolved |
|---|---|---|
| 0 | 0 | |
| 1 | 19.1 | |
| 2 | 36.8 | <30% |
| 3 | 47.8 | |
| 4 | 55.8 | 30-55% |
| 8 | 72.9 | 55-80% |
| 12 | 83.5 | 65-90% |
| 24 | 101.4 | >80% |

### EXAMPLE 5

Tablet composition comprising venlafaxine base

Tablet formulation

| | | | |
|---|---|---|---|
| Venlafaxine base | 30.00 | mg | Active substance |
| Silicified microcrystalline cellulose | 15.00 | mg | Filler |
| Anhydrous dicalcium phosphate | 44.10 | mg | Filler |
| Magnesium stearate | 0.90 | mg | Lubricant |
| Total weight | 90.00 | mg | |

Enteric coating formulation:

| | | |
|---|---|---|
| Sureteric | csp. | Gastroresistant |
| | | polymer |

Outlayer venlafaxine formulation:

| | | | |
|---|---|---|---|
| Venlafaxine base | 7.50 | mg | Active substance |
| HPMC | 2.30 | mg | Binder |
| PEG 6000 | 0.23 | mg | Plasticizer |
| Simethicone emulsion 30% USP | | q.s. | Antifoam agent |
| Purified water | | q.s. | |
| Total weight | 10.03 | mg | |

Modus operandi: Direct compression, enteric coating and venlafaxine base out coating Compression (Eccentric compression machine)

This product is compressed in round biconvex tablets of 5 mm diameter.

Enteric Coating (Coating Machine)

These tablets are coated with an enteric film made of Sureteric®.

A second film is made with the venlafaxine base outer layer and the coating process is carried out.

### EXAMPLE 6

Tablet composition comprising venlafaxine base

| | | | |
|---|---|---|---|
| Venlafaxine base | 30.00 | mg | Active substance |
| Glyceryl behenate (Compritol ATO 888) | 13.50 | mg | Fusible carrier |
| Microcrystalline cellulose | 42.0 | mg | Filler |
| Talc | 3.60 | mg | Antiadherent |
| Magnesium stearate | 0.90 | mg | Lubricant |
| Total weight | 90.00 | mg | |

Ratio Venlafaxine base: Compritol 1:0.45

Modus operandi: Hot melt granulation followed by compression.

Hot Melt Granulation (High Shear Mixer)

Venlafaxine base and Compritol ATO 888 are added to the bowl of the high shear mixer and mixed for 5 minutes. Bowl temperature is increased by hot air and microwaves up to approximately 60° C. and a partially melted mass of Compritol ATO 888 and Venlafaxine base is obtained. Then, hot air and microwaves are stopped and cool water is passed through the jacket, the melted product is cooled. This solid product is a granulate.

Sieving

This granulate obtained is sieved in order to calibrate the size particle.

Compression (Eccentric Compression Machine)

Microcrystalline cellulose is added to the granulate and mixed for 15 minutes. Talc and magnesium stearate are then added and mixed for 5 minutes. This product is compressed in round biconvex tablets of 5 mm diameter.

Dissolution rate (12 hours in SIF pH 6.8, 37° C., basket 100 rpm) was determined by UV spectrophotometry and is expressed in % of the declared amount.

| Hours | % dissolved | target % dissolved |
|---|---|---|
| 0 | 0 | |
| 1 | 11.3 | |
| 2 | 20.5 | <30% |
| 4 | 38.2 | 30-55% |
| 8 | 62.2 | 55-80% |
| 12 | 83.6 | 65-90% |

### EXAMPLE 7

Tablet composition comprising venlafaxine base

| | | | |
|---|---|---|---|
| Venlafaxine base | 37.50 | mg | Active substance |
| Glyceryl behenate (Compritol ATO 888) | 15.00 | mg | Fusible carrier |
| Lactose | 36.6 | mg | Filler |
| Magnesium stearate | 0.90 | mg | Lubricant |
| Total weight | 90.00 | mg | |

Ratio Venlafaxine base: Compritol 1:0.40

Modus operandi: Hot melt granulation followed by compression of the formed solid dispersion.

Venlafaxine Solid Dispersion by Hot Melt Granulation (High Shear Mixer)

Venlafaxine base and Compritol ATO 888 are added to the bowl of the high shear mixer and mixed for 5 minutes. Bowl temperature is increased by hot air and microwaves up to approximately 60° C. and a partially melted mass of Compritol ATO 888 and Venlafaxine base is obtained. Then, hot air and microwaves are stopped and cool water is passed through the jacket, the melted product is cooled. This solid product is a granulate.

Sieving

This granulate obtained is sieved in order to calibrate the size particle.

Compression (Eccentric Compression Machine)

Microcrystalline cellulose is added to the granulate and mixed for 15 minutes. Talc and magnesium stearate are then added and mixed for 5 minutes. This product is compressed in round biconvex tablets of 5 mm diameter.

Dissolution rate (12 hours in SIF pH 6.8, 37° C., basket 100 rpm) was determined by UV spectrophotometry and is expressed in % of the declared amount.

| Hours | % dissolved | Target % dissolved |
|---|---|---|
| 0 | 0 | |
| 1 | 22.9 | |
| 2 | 33.6 | <30% |
| 4 | 47.7 | 30-55% |
| 8 | 65.6 | 55-80% |
| 12 | 77.7 | 65-90% |
| 24 | 99.2 | >80 |

### EXAMPLE 8

Extended release composition with calcium phosphate filler

Composition of the tablet:

| | |
|---|---|
| Ingredients | Quantity |

| | | |
|---|---|---|
| Venlafaxine base | 37.5 | mg |
| A-tab (anhydrous dicalcium phosphate) | 41.70 | |
| Magnesium stearate | 0.80 | mg |
| Tablet weight | 80.0 | mg |

Modus operandi: direct compression

Dissolution profile:

| | % dissolved | Target |
|---|---|---|
| Time (hours) | SIF pH 6.8 | SIF pH 6.8 |
| 2 | 29.7 | <30% |
| 4 | 41.7 | 30-55% |
| 8 | 57.3 | 55-80% |
| 12 | 67.9 | 65-90% |
| 24 | 87.5 | >80% |

### EXAMPLE 9

Extended release tablet with calcium phosphate and PVP

Composition:

| | |
|---|---|
| Ingredients | Quantity |

| | | |
|---|---|---|
| Venlafaxine base | 37.5 | mg |
| Polyvinylpyrrolidone | 2.25 | mg |
| A-tab (anhydrous dicalcium phosphate) | 39.45 | mg |
| Magnesium stearate | 0.80 | mg |
| Tablet weight | 80.0 | mg |

Modus operandi: Wet granulation of venlafaxine with Polyvinylpyrrolidone (PVP)

Blending the granulate with other excipient and compressing

The dissolution profile is:

| | % Dissolved | Product Specifications |
|---|---|---|
| Time (hours) | SIF pH 6.8 | SIF pH 6.8 |
| 2 | 29.4 | <30% |
| 4 | 42.6 | 30-55% |
| 8 | 59.4 | 55-80% |
| 12 | 70.8 | 65-90% |
| 24 | 80.7 | >80% |

### EXAMPLE 10

Extended release tablet with low content of the filler

Composition

| | |
|---|---|
| Ingredients | Quantity |

| | | |
|---|---|---|
| Venlafaxine base | 37.5 | mg |
| Polyvinylpyrrolidone | 2.25 | mg |
| A-tab (anhydrous dicalcium phosphate) | 19.65 | mg |
| Magnesium stearate | 0.60 | mg |
| Tablet weight | 60.0 | mg |

Modus operandi: Venlafaxine and PVP were blended together by wet granulation

The granulate was mixed with remaining excipients and compressed into tablets.

Dissolution profile:

| | % Dissolved | Target % Dissolved |
|---|---|---|
| Time (hours) | SIF pH 6.8 | SIF pH 6.8 |
| 2 | 28.8 | <30% |
| 4 | 42.6 | 30-55% |
| 8 | 59.2 | 55-80% |
| 12 | 70.5 | 65-90% |
| 24 | 80.6 | >80% |

### EXAMPLE 11

Extended release tablet with calcium phosphate, PVP and Compritol

Composition

| | |
|---|---|
| Ingredients | Quantity |

| | | |
|---|---|---|
| Venlafaxine base | 37.5 | mg |
| Polyvinyl pyrrolidone | 2.25 | mg |
| A-tab (anhydrous dicalcium phosphate) | 19.73 | mg |
| Compritol ATO 888 (Glyceryl behenate) | 19.73 | mg |
| Magnesium stearate | 0.80 | mg |
| Tablet weight | 80.0 | mg |

Modus operandi: Venlafaxine and PVP were blended together by wet granulation

The granulate was mixed with remaining excipients and compressed into tablets.

Dissolution profile:

| | % Dissolved | Target % Dissolved |
|---|---|---|
| Time (hours) | SIF pH 6.8 | SIF pH 6.8 |
| 2 | 22.3 | <30% |
| 4 | 32.6 | 30-55% |
| 8 | 46.7 | 55-80% |
| 12 | 57.2 | 65-90% |
| 24 | 77.5 | >80% |

### EXAMPLE 12

Molecular dispersion of venlafaxine base with polyvinylpyrrolidone 2 g venlafaxine base was dissolved in 15 ml ethanol and a turbid solution was obtained. 2 g of polyvinylpyrrolidone (Polivydon 25) was added and the solvent was removed by drying in an oven at 60° C. during 15 hours. A solid particulate product was obtained.

A milled sample was analyzed by Differential Scanning Calorimetry in comparison with venlafaxine base, polyvinylpyrrolidone and a physical mixture of venlafaxine base and polyvinylpyrrolidone (1:1 ratio). Results obtained by DSC: Venlafaxine base: melting endotherm peak appears at approximately 79° C., PVP: no melting peak up to 150° C. Physical mixture: endotherm peak at approx. 77° C. Solid dispersion: no endotherm up to 150° C.

### EXAMPLE 13

Extended release tablets with venlafaxine base, calcium phosphate and glyceryl behenate

Tablet composition:

| | |
|---|---|
| Ingredients | Quantity |

| | | |
|---|---|---|
| Venlafaxine base | 37.5 | mg |
| Compritol ATO 888 (glyceryl behenate) | 20.45 | mg |
| Dicafos AN or Dicafos A (dicalcium | 20.45 | mg |
| phosphate) | | |
| Magnesium stearate | 0.80 | mg |
| Aerosil 200 | 0.80 | mg |
| Tablet weight | 80.0 | mg |

Manufacturing process:

direct compression

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

We claim:

1. A pharmaceutical composition comprising a solid venlafaxine base and a pharmaceutically acceptable excipient.

2. The pharmaceutical composition according to claim 1, wherein said composition is an extended release composition.

3. The pharmaceutical composition according to claim 2, wherein said excipient is selected from the group consisting of calcium phosphates, polymers, waxes, sugars, and combinations thereof.

4. The composition according to claim 3, wherein said at least one excipient is selected from the group consisting of HPMC, microcrystalline cellulose, polyvinylpyrrolidone, and calcium phosphates.

5. The composition according to claim 4, which further comprises a lubricant.

6. The composition according to claim 3, wherein said at least one excipient is selected from the group consisting of hydrogenated castor oil, glyceryl behenate, glycerylpalmito stearate, and saturated polyglycolyzed glycerate.

7. The pharmaceutical composition according to claim 1, wherein said composition is in the form of granules or pellets.

8. The pharmaceutical composition according to claim 2, wherein said composition is in the form of a tablet.

9. The pharmaceutical composition according to claim 8, wherein said composition is a unit dosage form and said venlafaxine is contained in an amount between 30 mg and 300 mg.

10. The composition according to claim 8, wherein said composition is a once daily dose tablet.

11. The composition according to claim 8, wherein said composition has a dissolution profile such that less than 30% of said venlafaxine is released from said composition in 2 hours using purified water at 37° C. with stirring at 100 r.p.m. in a USP I (basket) apparatus.

12. The composition according to claim 11, wherein said composition has a release profile that satisfies the following

| Time (hours) | Average % venlafaxine released |
|---|---|
| 2 | <30 |
| 4 | 30-55 |
| 8 | 55-80 |
| 12 | 65-90 |
| 24 | >80 |

using USP Apparatus 1 (basket) at 100 rpm in purified water at 37° C.

13. The composition according to claim 2, wherein said composition is a tablet and said at least excipient is a matrix material.

14. The composition according to claim 13, wherein said matrix material is a hydrophilic, lipophilic or biodegradable matrix material.

15. The composition according to claim 14, wherein said matrix material is a lipophilic matrix material.

16. The composition according to claim 15, wherein said matrix material is selected from the group consisting of glyceryl palmitostearate, glyceryl behenate, and hydrogenated castor oil.

17. The composition according to claim 16, wherein said tablet further comprises a calcium phosphate, a lubricant, or both.

18. The composition according to claim 16, wherein said tablet is a once daily dose tablet.

19. The composition according to claim 1, wherein said composition is in the form of pellets.

20. The composition according to claim 19, wherein said composition is a once daily dose capsule containing said pellets.

21. The composition according to claim 1, wherein said venlafaxine base is contained in an amount of at least 40 wt %.

22. A venlafaxine composition comprising venlafaxine base dispersed in a solid carrier.

23. The venlafaxine composition according to claim 22, wherein said venlafaxine is in a molecular dispersion within said carrier.

24. The venlafaxine composition according to claim 22, wherein said carrier is selected from a polymer and a fatty acid wax.

25. The venlafaxine composition according to claim 24, wherein said excipient is selected from polyvinylpyrrolidone, glyceryl palmitostearate, glyceryl behenate, and hydrogenated castor oil.

26. The venlafaxine composition according to claim 25, wherein said composition is in the form of granules.

27. A method for treating a venlafaxine-treatable disease or condition, which comprises administering to a patient in need thereof an effective amount of the composition according to claim 1.

28. The method according to claim 27, wherein said patient suffers from depression and said effective amount of venlafaxine base is an antidepressant amount.

29. The method according to claim 27, wherein said composition is administered once daily.

30. The method according to claim 29, wherein said composition is administered in the form of one or two tablets.

31. A process, which comprises dispersing venlafaxine base in a liquid-phase carrier; and solidifying said liquid phase to form a solid dispersion of venlafaxine.

32. The process according to claim 31, wherein said dispersing step comprises mixing venlafaxine base and a molten fusible carrier to form at least a partially melted mass; and said solidifying step comprises cooling said at least partially melted mass to form a solidified product.

33. The process according to claim 32, wherein said solidified product is in the form of granules or pellets.

34. The process according to claim 33, which further comprises milling said solidified product to form granules.

35. The process according to claim 32, which further comprises combining, prior to said mixing step, said fusible carrier in a non-molten state with said venlafaxine and heating to render said fusible carrier molten.

36. The process according to claim 35, wherein said fusible carrier is a lipophilic matrix material.

37. The process according to claim 36, wherein said fusible carrier is a wax.

38. The process according to claim 32, wherein said mixing step further includes mixing at least one excipient selected from the group consisting of calcium phosphates, microcrystalline cellulose, and lactose.

39. The process according to claim 33, which further comprises mixing said solidified product, optionally after milling, with a lubricant and at least one excipient selected from the group consisting of calcium phosphates, microcrystalline cellulose, and lactose.

40. The process according to claim 31, wherein said liquid-phase excipient is a polymer dissolved in a solvent and said solidifying step comprises removing said solvent.

41. The process according to claim 40, wherein said polymer is polyvinylpyrrolidone.

42. The process according to claim 31, which further comprises converting said solidified product into a tablet.

43. The process according to claim 42, wherein said tablet is an extended release tablet.
